(19) United States
(12) Patent Application Publication
(10) Pub. No.: US 2005/0124941 A1
(43) Pub. Date: Jun. 9, 2005

(54) Package for pharmaceutical formulation

(76) Inventors: Martin Panchula, Vacaville, CA (US); Stan Lam, Dublin, CA (US); Pete McArthur, Danville, CA (US); Scott Lautenbach, San Mateo, CA (US)

Correspondence Address: WOODCOCK WASHBURN LLP, ONE LIBERTY PLACE, 46TH FLOOR, PHILADELPHIA, PA, 19103, US

(21) Appl. No.: 10/988,020

(22) Filed: Nov. 12, 2004

Related U.S. Application Data
(60) Provisional application No. 60/520,547, filed on Nov. 14, 2003.

(51) Int. Cl. A61M 5/315

(52) U.S. Cl. 604/218

(57) ABSTRACT

Cartridges are provided for storing and facilitating the delivery of pharmaceutical formulations. In one preferred embodiment, the cartridge comprises a body including a bore extending through the body, and a plunger movably disposed in the bore. The bore has a transverse dimension at a distal end that is equivalent to that at a midpoint. The plunger has a planar contact surface that is transversely coextensive with the bore for applying a force to a pharmaceutical formulation contained in the bore. This configuration helps to eliminate dead volume, and therefore enables filing by volume rather than weight to ensure accurate dosing.

### RELATED APPLICATION

This application claims the benefits of U.S. Provisional Application No. 60/520,547, filed on Nov. 14, 2003, which is incorporated herein by reference.

### FIELD OF THE INVENTION

The present invention relates generally to packages for storing and facilitating the delivery of parenteral pharmaceutical formulations. At least some of the preferred embodiments are particularly suitable for use with highly viscous, depot-type compositions.

### BACKGROUND OF THE INVENTION

Parenteral pharmaceutical formulations are typically based on either lyophilized cake, which is dissolved immediately prior to use, solutions, or dispersions. Traditional syringes with Luer-type fittings are typically used for delivering parenteral formulations. The traditional syringes generally consist of a barrel and a plunger. The barrel has a bore for receiving a pharmaceutical formulation. The bore at the distal end tends to neck down to accommodate a Luer fitting.

Traditional syringes can be incompatible with high viscosity formulations. Bore necking (tapering) can create a void or dead volume, leading to the introduction of air into the system, which can be difficult to purge when the parenteral formulation has a high viscosity. A tapered bore can also result in a portion of the pharmaceutical formulation being left behind in the syringe barrel. It can be particularly difficult to completely expel a high viscosity formulation from a tapered bore syringe. Furthermore, a Leur fitting or closure generally does not serve as an adequate microbial barrier to a contained pharmaceutical formulation. Grip flanges and plungers associated with traditional syringes also increase the volume of secondary packaging and the per unit volume required for shipping the product.

Accordingly, there are packaging and delivery needs for high viscosity pharmaceutical formulations that are unrecognized and unmet with the state of the art.

### SUMMARY OF THE INVENTION

The present invention is directed to packages that are useful for storing and dispensing pharmaceutical formulations. In accordance with one preferred embodiment of the present invention, there has now been provided a cartridge comprising a body including a bore extending through the body, a pharmaceutical formulation disposed within a portion of the bore, and a plunger movably disposed within the bore for expelling the pharmaceutical formulation. The pharmaceutical formulation has a viscosity of from about 1,000 to about 5,000 poise. The plunger has a length that is shorter than that of the bore.

In accordance with another preferred embodiment of the present invention, there has now been provided a cartridge comprising a body including a bore extending therethrough, and a plunger movably disposed in the bore. The bore has a transverse dimension at a distal end that is equivalent to that at a midpoint. The plunger has a planar contact surface that is transversely coextensive with the bore for applying a force to a pharmaceutical formulation contained in the bore.

In accordance with yet another preferred embodiment of the present invention, there has now been provided a cartridge comprising a body including a distal end and a wall of varying thickness, and a bore extending through the body for receiving a pharmaceutical formulation. Relative to positions other than the distal end, the wall thickness of the body at the distal end is reduced while the transverse dimension of the bore at the distal end is unchanged.

The present invention is also directed to methods for delivering pharmaceutical formulations. In accordance with one preferred method embodiment of the present invention, there has now been provide a method comprising a first step of providing a cartridge having a body including a bore extending through the body, a pharmaceutical formulation disposed within a portion of the bore, and a plunger movably disposed within the bore for expelling the pharmaceutical formulation. The pharmaceutical formulation has a viscosity of from about 1,000 to about 5,000 poise. The plunger has a length that is shorter than that of the bore. The plunger is engaged with a separately formed rod. And the plunger is transferred from a first position to a second position by applying a normal force to the rod, such that the pharmaceutical formulation is expelled from the cartridge.

In accordance with another preferred method embodiment of the present invention, a cartridge is provided comprising a body including a bore extending through the body, and an end surface having an opening therein that is in fluid communication with the bore. A pharmaceutical formulation is disposed within a portion of the bore, and a plunger is movably disposed within the bore for expelling the pharmaceutical formulation. The plunger is transferred from a first position to a second position, such that a contact surface of the plunger in the second position is flush with, or extends slightly beyond, the body end surface.

### BRIEF DESCRIPTION OF THE DRAWINGS

The present invention is believed to be best understood through the following detailed description of the illustrative embodiments and the accompanying drawings wherein like reference numerals indicate like features, and wherein:

FIG. 1 is a perspective view of a preferred cartridge embodiment provided by the present invention;

FIG. 2 is a cross-sectional view of the cartridge embodiment shown in FIG. 1 taken along line 2-2;

FIG. 3 is a cross-sectional view of the cartridge embodiment shown in FIG. 1 including a pharmaceutical formulation disposed in its bore, a plunger positioned behind the formulation, and a seal covering an opening in the distal end of the cartridge body;

FIG. 4 is perspective view of a preferred plunger embodiment, in accordance with the present invention, having a recess formed in an engagement surface, and

FIGS. 5A-5C is a series of cross-sectional views illustrating a preferred method of delivering a pharmaceutical formulation as provided by the present invention.

### DETAILED DESCRIPTION OF ILLUSTRATIVE EMBODIMENTS

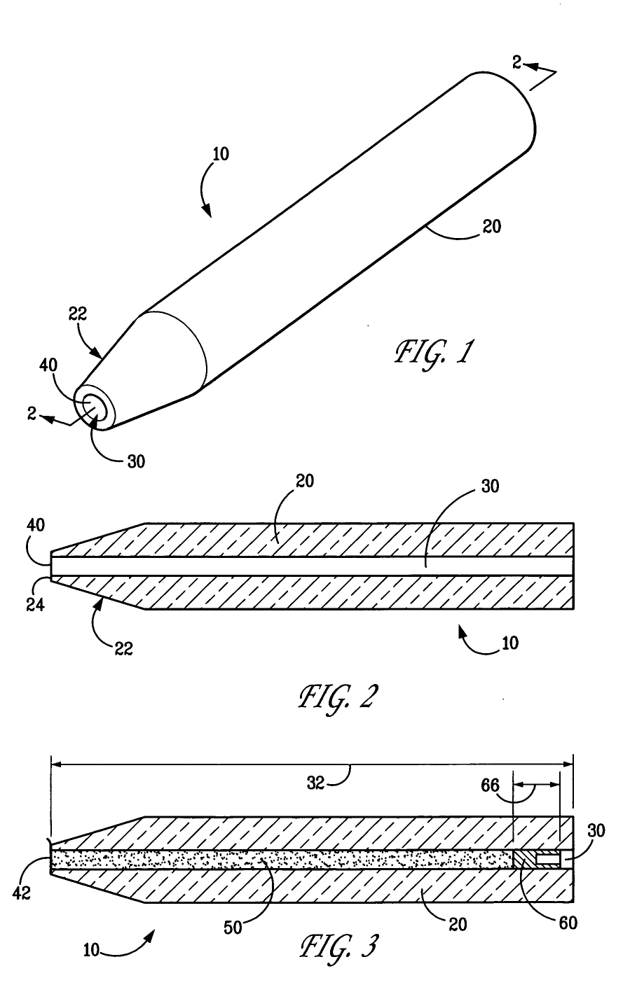

Referring now to the drawings, wherein like reference numerals designate like structure throughout the views, and referring in particular to FIGS. 1 and 2, a cartridge 10 is shown having a body 20 with distal end 22, and a bore 30 extending through body 20. Body 20 can be made from any material that is compatible with pharmaceutical applications and that is capable of withstanding high pressure (e.g., internal pressures of approximately 500 to 2000 p.s.i., and higher) without substantial deformation. A representative, non-limiting, list of materials for body 20 includes metals, such as stainless steel, aluminum, and titanium; various glasses, and plastics, such as HDPE, UHDPE, acetyls, fluoropolymers, and other engineering plastics, meeting specific drug product requirements. Preferably, body 20 is a glass tube made from a type I glass material. An opening 40 is formed in an end surface 24 that is fluidly connected with bore 30. A pharmaceutical formulation is preferably both introduced and expelled through opening 40.

FIG. 3 illustrates cartridge 10 in a loaded and sealed configuration, with a pharmaceutical formulation 50 disposed in a portion of bore 30, a first seal 42 covering opening 40, and a plunger 60 positioned behind formulation 50 to effect a second seal. In this configuration, the pre-filled cartridge accordingly is a package for storing and transporting a pharmaceutical formulation prior to use with a patient. When ready for administration, the cartridge can be manipulated with appropriate associated devices to expel the pharmaceutical formulation via opening 40.

Seal 42 is coupled to end surface 24 after filing bore 30 with a pharmaceutical formulation 50. Seal 42 is preferably made from a sheet of material consisting of one or more layers and serves as a primary microbial barrier. Suitable materials include, but are not limited to, polymeric films, metal foils, and laminations thereof. Seal 42 can be coupled to end surface 24 through heat or induction such that the cartridge is hermetically sealed. In some embodiments, seal 42 is designed and configured for removal prior to expelling a contained pharmaceutical formulation. In other embodiments, seal 42 is frangible and permanently coupled to end surface 24, whereby seal 42 is pierced with an appropriate device just prior to or simultaneously with formulation expulsion. A frangible seal eliminates extra handling prior to use, which not only simplifies formulation delivery, but also reduces the potential for contamination when used with a suitable outer covering.

In a preferred embodiment, and as shown in the figures, bore 30 is straight (not tapered) along its entire length. That is, the transverse dimension of bore 30 is constant. In alternative embodiments, bore 30 may have a transverse dimension that varies along its length. However in such embodiments, bore portions located, for example, from at least distal end 22 up to approximately a midpoint should preferably remain straight. A straight, non-tapered bore 30 at distal end 22 eliminates the existence of void or dead volume that can potentially introduce air into the cartridge. Incorporated air is difficult to purge and can compromise and/or complicate accurate dosing by, for example, increasing the compressibility of the pharmaceutical formulation. A straight bore 30 can also simplify and decrease the cost of manufacturing cartridge 10. In preferred embodiments, the transverse dimension of bore 30 is from about 2 to about 10 mm.

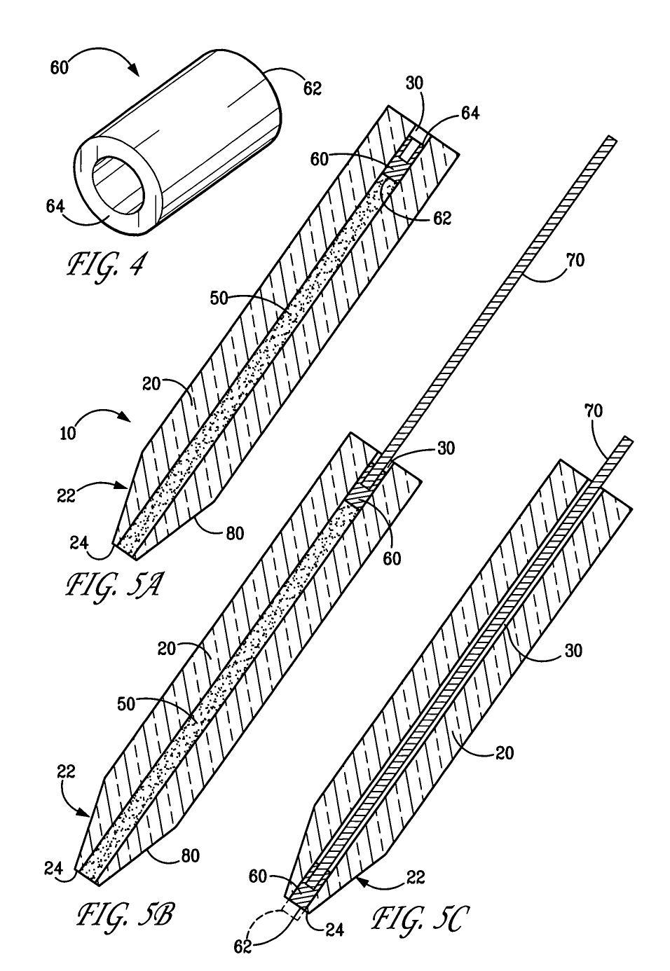

Plunger 60 is movably disposed in bore 30, and includes a planar contact surface 62 that is sized to be transversely coextensive with bore 30. The combination of a planar contact surface 62 and a straight bore 30 (at distal end 22) permits positioning plunger 60 in a flush arrangement with end surface 24 before filing. This arrangement eliminates any dead volume and enables filing by volume rather than weight to help ensure accurate dosing. Plunger 60 has a length 66 that is shorter than a length 32 of bore 30, and is preferably devoid of any member extending beyond cartridge body 20. Thus, the amount of secondary packaging and the shipping volume associated with cartridge 10 is minimized. A separately formed rod or other device engages an engagement end 64 of plunger 60 for altering the plunger's position within bore 30 and for expelling a contained pharmaceutical formulation 50. As best shown in FIG. 4, engagement end 64 has an optional recess formed therein for positively receiving a rod. Since a single rod can be used with multiple cartridges, the costs of delivering pharmaceutical formulations stored in cartridges 10 is reduced. In preferred embodiments, plunger 60 is made out of TEFLON, HDPE, rubber formulations, or a combination of such materials. Plunger 60 may be made from other materials known by those having ordinary skill in the art.

Figure series 5A-5C illustrates a pharmaceutical formulation being delivered from a pre-filled cartridge 10. In FIG. 5A, a pharmaceutical formulation 50 resides within a portion of bore 30 and a plunger 60 is located in a first position behind the formulation. Note that a seal previously covering opening 40 has been removed. Next, and as can be seen in FIG. 5B, a rod 70 engages plunger 60. A normal force is applied to rod 70 to transfer plunger 60 from its first position to a second position at the cartridge body distal end 22 (shown in FIG. 5C), such that the pharmaceutical formulation is expelled form cartridge 10. To ensure that the pharmaceutical formulation 50 is completely expelled, the contact surface 62 of plunger 60 in the second position is preferably at least flush with body end surface 24. The contact surface 62 may also extend slightly beyond end surface 24 as is shown with broken lines.

The distal end 22 of cartridge 10 will generally be seated into an applicator tip (e.g., in the form of a needle device or catheter device) prior to applying a normal force to rod 70. An outer surface 80 at the distal end 22 of body 20 is preferably angled (e.g., beveled or tapered radially inwardly), such that a seal can automatically be formed between cartridge 10 and the applicator tip to prevent “blow-back” of a high viscosity pharmaceutical formulation as it exits opening 40. One way of providing an angled distal end 22 is by reducing the wall thickness of body 20 at the distal end. Distal end 22 is shown in the figures having a single taper. However, distal end 22 may have multiple tapers, stepped portions, or any other geometrical variation resulting in a reduction of wall thickness while maintaining a straight, non-tapered bore.

Cartridges of the present invention can be pre-filled with a variety of parenteral pharmaceutical formulations having both low and high viscosity values. Viscosity values typically range from about 100 to about 500,000 poise, and more particularly from about 1,000 to about 5,000 poise. Viscosity values can be measured at a 0.1 sec⁻¹ shear rate and 25° C. using a Haake Rheometer at about 1-2 days after formulation makeup is completed. Preferred cartridges, as described above, are particularly suitable for storing and facilitating the delivery of high viscosity formulations, including, but not limited to, gel-like depot compositions. Exemplary depot compositions generally include a beneficial agent dispersed or dissolved in a gel vehicle made up of a polymer and a solvent. A discussion of individual components of exemplary depot compositions follows. A more detailed discussion of exemplary depot compositions is disclosed in U.S. patent application Ser. No. 10/628,984, filed Jul. 28, 2003, which is incorporated by reference herein.

Polymers of exemplary depot compositions gradually hydrolyze, dissolve, physically erode, or otherwise disintegrate within the aqueous fluids of a patient's body. Generally, the polymers bioerode as a result of hydrolysis or physical erosion, although the primary bioerosion process is typically hydrolysis. Such polymers include, but are not limited to, polylactides, polyglycolides, polycaprolactones, polyanhydrides, polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, polyhydroxycellulose, chitin, chitosan, hyaluronic acid and copolymers, terpolymers and mixtures thereof. Preferred polymers are polylactides, that is, a lactic acid-based polymer that can be based solely on lactic acid or can be a copolymer based on lactic acid and glycolic acid, and which may include small amounts of other comonomers. As used herein, the term “lactic acid” includes the isomers L-lactic acid, D-lactic acid, DL-lactic acid and lactide, while the term “glycolic acid” includes glycolide. Most preferred are poly(lactide-co-glycolide)copolymers, commonly referred to as “PLGA.”0 The polymer may have a monomer ratio of lactic acid/glycolic acid of from about 100:0 to about 15:85, preferably from about 75:25 to about 30:70, more preferably from about 60:40 to about 40:60, and an especially useful copolymer has a monomer ratio of lactic acid/glycolic acid of about 50:50.

Suitable lactic acid-based polymers are available commercially. For instance, 50:50 lactic acid:glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below. Examples of polymers include, but are not limited to, Poly (D,L-lactide) Resomer® L104, PLA-L104, code no. 33007, Poly (D,L-lactide-co-glycolide) 50:50 Resomer® RG502, code 0000366, Poly (D,L-lactide-co-glycolide) 50:50 Resomer® RG502H, PLGA-502H, code no. 260187, Poly (D,L-lactide-co-glycolide) 50:50 Resomer® RG503, PLGA-503, code no. 0080765, Poly (D,L-lactide-co-glycolide) 50:50 Resomer® RG506, PLGA-506, code no. 95051, Poly (D,L-lactide-co-glycolide) 50:50 Resomer® RG755, PLGA-755, code no. 95037, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly glycolide (Resomer® G 205); Poly D,L-lactide-co-glycolide 50:50 (Resomer® RG 504 H, Resomer® RG 504, Resomer® RG 505); Poly D-L-lactide-co-glycolide 75:25 (Resomer® RG 752, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A(3), MEDISORB® Polymer 5050 DL 3A(3), MEDISORB® Polymer 5050 DL 4A(3)) (Medisorb Technologies International L.P., Cincinatti, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ε-caprolactone; Poly DL-lactide-co-caprolactone 25:75; and Poly DL-lactide-co-caprolactone 75:25 (Birmingham Polymers, Inc., Birmingham, Ala.).

A polymer matrix may alternatively be used in the exemplary depot compositions, comprising a plurality of bioerodible, biocompatible polymers wherein each polymer of the plurality of polymers has a specified weight average molecular weight; the polymer matrix having a broad molecular weight distribution of the plurality of polymers. Preferably, the polymer matrix has a multi-modal molecular weight distribution of a plurality of polymers; wherein a first of the plurality of polymers is a low molecular weight (LMW) polymer; a second of the plurality of polymers is a high molecular weight (HMW) polymer; and optionally a third of the plurality of polymers is a medium molecular weight (MMW) polymer; each polymer having a polydispersity of at least 2. The polymer or polymer matrix is generally present in depot compositions in an amount ranging from about 5 to about 90% by weight, and preferably from about 35 to about 75% by weight.

The second component of the gel vehicle typically used in depot compositions is a water-immiscible solvent preferably having a miscibility in water that is less than 7% by weight at 25° C. The solvent must be biocompatible, should form a gel, preferably a viscous gel with the polymer, and restrict water uptake. The solvent is preferably selected from the group consisting of an aromatic alcohol, esters of aromatic acids, aromatic ketones, and mixtures thereof. Most preferred solvents are derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with benzyl benzoate being most especially preferred.

Exemplary depot compositions may also include, in addition to the water-immiscible solvent(s), one or more additional miscible solvents (“component solvents”), provided that any such additional solvent is other than a lower alkanol. Component solvents compatible and miscible with the primary solvent(s) may have a higher miscibility with water and the resulting mixtures may still exhibit significant restriction of water uptake into the implant. Such mixtures will be referred to as “component solvent mixtures.” Useful component solvent mixtures may exhibit solubilities in water greater than the primary solvents themselves, typically between 0.1 weight percent and up to and including 50 weight percent, preferably up to and including 30 weight percent, and most preferably up to an including 10 weight percent, without detrimentally affecting the restriction of water uptake exhibited by the implants of the invention. Component solvents useful in component solvent mixtures are those solvents that are miscible with the primary solvent or solvent mixture, and include, but are not limited, to triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glycerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, and 1-dodecylazacyclo-heptan-2-one, and mixtures thereof.

The solvent or solvent mixture is typically present in an amount of from about 95 to about 5% by weight, preferably about 75 to about 15% by weight, and most preferably about 65% to about 20% by weight of the viscous gel. The solvent or solvent mixture is capable of dissolving the polymer to form a viscous gel that can maintain particles of the beneficial agent dissolved or dispersed and isolated from the environment of use prior to release.

A beneficial agent is generally dissolved or dispersed in the gel vehicle formed from the polymer and solvent. The beneficial agent is preferably incorporated into the viscous gel in the form of particles typically having an average particle size of from about 0.1 to about 250 microns. The beneficial agent is typically dissolved or dispersed in the composition in an amount of from about 0.1% to about 50% by weight, preferably in an amount of from about 1% to about 30%, more preferably in an amount of about 2% to about 20%, and often 2 to 10% by weight of the combined amounts of the polymer mixture, solvent, and beneficial agent.

The beneficial agent can be any physiologically or pharmacologically active substance or substances optionally in combination with pharmaceutically acceptable carriers and additional ingredients such as antioxidants, stabilizing agents, permeation enhancers, etc. that do not substantially adversely affect the advantageous results that can be attained by the present invention. The beneficial agent may be any of the agents which are known to be delivered to the body of a human or an animal and that are preferentially soluble in water rather than in the polymer-dissolving solvent. These agents include drug agents, medicaments, vitamins, nutrients, or the like. Included among the types of agents which meet this description are lower molecular weight compounds, proteins, peptides, genetic material, nutrients, vitamins, food supplements, sex sterilants, fertility inhibitors and fertility promoters.

Beneficial agents include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, chemotherapeutic agents, immunosuppressive agents, anti-inflammatory agents including anti-inflammatory corticosteroids, antiproliferative agents, antimitotic agents, angiogenic agents, antipsychotic agents, central nervous system (CNS) agents, anticoagulants, fibrinolytic agents, growth factors, antibodies, ocular drugs, and metabolites, analogs (including synthetic and substituted analogs), derivatives (including aggregative conjugates/fusion with other macromolecules and covalent conjugates with unrelated chemical moieties by means known in the art) fragments, and purified, isolated, recombinant and chemically synthesized versions of these species.

Particular drugs include, but are not limited to, procaine, procaine hydrochloride, tetracaine, tetracaine hydrochloride, cocaine, cocaine hydrochloride, chloroprocaine, chloroprocaine hydrochloride, proparacaine, proparacaine hydrochloride, piperocaine, piperocaine hydrochloride, hexylcaine, hexylcaine hydrochloride, naepaine, naepaine hydrochloride, benzoxinate, benzoxinate hydrochloride, cyclomethylcaine, cyclomethylcaine hydrochloride, cyclomethylcaine sulfate, lidocaine, lidocaine hydrochloride, bupivicaine, bupivicaine hydrochloride, mepivicaine, mepivacaine hydrochloride, prilocaine, prilocaine hydrochloride, dibucaine and dibucaine hydrochloride, etidocaine, benzocaine, propoxycaine, dyclonin, pramoxine, oxybuprocaine, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-nor-progesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat, captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth factors such as epidermal growth factors (EGF), platelet-derived growth factors (PDGF), fibroblast growth factors (FGF), transforming growth factors-α (TGF-α), transforming growth factors-β (TGF-β), erythropoietin (EPO), insulin-like growth factor-I (IGF-I), insulin-like growth factor-II (IGF-II), interleukin-1, interleukin-2, interleukin-6, interleukin-8, tumor necrosis factor-α (TNF-α), tumor necrosis factor-β, (TNF-β), Interferon-α (INF-α), Interferon-β (INF-β), Interferon-γ (INF-γ), Interferon-ω (INF-ω), colony stimulating factors (CGF), vascular cell growth factor (VEGF), thrombopoietin (TPO), stromal cell-derived factors (SDF), placenta growth factor (P1GF), hepatocyte growth factor (HGF), granulocyte macrophage colony stimulating factor (GM-CSF), glial-derived neurotropin factor (GDNF), granulocyte colony stimulating factor (G-CSF), ciliary neurotropic factor (CNTF), bone morphogeneic proteins (BMP), coagulation factors, human pancreas hormone releasing factor, analogs and derivatives of these compounds, and pharmaceutically acceptable salts of these compounds, or their analogs or derivatives.

Additional examples of drugs that may be delivered by cartridges of the present invention include, but are not limited to, antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin, actinomycin D, daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP)II_{b}IIIₐ inhibitors and vitronectin receptor antagonists; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes—dacarbazinine (DTIC); antiproliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine (cladribine)); platinum coordination complexes (cisplatin, carboplatin), procarbazine, hydroxyurea, mitotane, aminoglutethimide; hormones (i.e. estrogen); antipsychotic agents, (such as antipsychotic drugs, neuroleptic drugs, tranquillisers and antipsychotic agents binding to dopamine, histamine, muscarinic cholinergic, adrenergic and serotonin receptors, including but not limited to phenothiazines, thioxanthenes, butyrophenones, dibenzoxazepines, dibenzodiazepines and diphenylbutylpiperidines); central nervous system (CNS) agents; anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); antiinflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen); indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blocker; nitric oxide donors; anti-sense oligionucleotides and combinations thereof; cell cycle inhibitors, mTOR inhibitors, and growth factor signal transduction kinase inhibitors, analogs and derivatives of these compounds, and pharmaceutically acceptable salts of these compounds, or their analogs or derivatives

Other beneficial agents include chemotactic growth factors, proliferative growth factors, stimulatory growth factors, and transformational peptide growth factors including genes, precursors, post-translational-variants, metabolites, binding-proteins, receptors, receptor agonists and antagonists of the following growth factor families: epidermal growth factors (EGFs), platelet-derived growth factor (PDGFs), insulin-like growth factors (IGFs), fibroblast-growth factors (FGFs), transforming-growth factors (TGFs), interleukins (ILs), colony-stimulating factors (CSFs, MCFs, GCSFs, GMCSFs), Interferons (IFNs), endothelial growth factors (VEGF, EGFS), erythropoietins (EPOs), angiopoietins (ANGs), placenta-derived growth factors (PlGFs), and hypoxia induced transcriptional regulators (HIFs).

Beneficial agents may also comprise chemotherapeutic agents for the local application of such agents to avoid or minimize systemic side effects. Representative chemotherapeutic agents include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986. To the extent not mentioned above, the beneficial agents described in aforementioned U.S. Pat. No. 5,242,910 can also be used.

It is to be understood that even though numerous characteristics and advantages of the present invention have been set forth in the foregoing description, together with details of the structure and finction of the invention, the disclosure is illustrative only. Accordingly, changes may be made in detail, especially in matters of shape, size and arrangement of features within the principles of the invention to the full extent indicated by the broad general meaning of the terms in which the appended claims are expressed.

1. A cartridge for storing and facilitating the delivery of a pharmaceutical formulation, the cartridge comprising:
a) a body including a bore extending therethrough, the bore having a bore length;
b) a pharmaceutical formulation disposed within a portion of the bore, the pharmaceutical formulation having a viscosity of from about 1,000 to about 5,000 poise; and
c) a plunger movably disposed within the bore for expelling the pharmaceutical formulation, the plunger having a length that is shorter than the bore length.

2. The cartridge of claim 1, wherein the bore has a transverse dimension that is constant along its entire length.

3. The cartridge of claim 1, further comprising a seal covering an opening in a distal end of the body that is in fluid communication with the bore.

4. The cartridge of claim 3, wherein the seal comprises a foil that is coupled to the body via heat or induction.

5. The cartridge of claim 1, wherein the pharmaceutical formulation comprises a parenteral formulation.

6. The cartridge of claim 1, wherein the body is a glass tube.

7. The cartridge of claim 6, wherein the glass tube is capable of withstanding an internal pressure of from about 500 to about 2,000 p.s.i.

8. The cartridge of claim 1, wherein the plunger is devoid of any member extending beyond the body.

9. The cartridge of claim 1, wherein the body has an angled outer surface at its distal end while a portion of the bore radially inward from the angled outer surface is straight.

10. The cartridge of claim 1, wherein an engagement end of the plunger includes a recess for accepting a rod.

11. A cartridge for storing and facilitating the delivery of a pharmaceutical formulation, the cartridge comprising:
a) a body including a bore extending therethrough, the bore having a transverse dimension at a distal end that is equivalent to that at a midpoint; and
b) a plunger movably disposed in the bore, the plunger including a planar contact surface that is transversely coextensive with the bore for applying a force to a pharmaceutical formulation contained in the bore.

12. The cartridge of claim 11, wherein the plunger has a length that is shorter than that of the bore.

13. The cartridge of claim 11, further comprising a pharmaceutical formulation disposed within a portion of the bore, wherein the pharmaceutical formation has a viscosity of from about 100 to about 500,000 poise.

14. The cartridge of claim 13, wherein the pharmaceutical formulation has a viscosity of from about 1,000 to about 5,000 poise.

15. The cartridge of claim 11, wherein the pharmaceutical formulation comprises a parenteral formulation.

16. The cartridge of claim 11, wherein the body has a tapered outer surface at its distal end.

17. The cartridge of claim 11, wherein an engagement end of the plunger includes a recess for accepting a rod.

18. A cartridge for storing and facilitating the delivery of a pharmaceutical formulation, the cartridge comprising:
a) a body including a distal end and a wall of varying thickness; and
b) a bore extending through the body for receiving a pharmaceutical formulation, the bore having a transverse dimension;
wherein, relative to positions other than the distal end, the wall thickness of the body at the distal end is reduced while the transverse dimension of the bore at the distal end is unchanged.

19. The cartridge of claim 18, further comprising a seal covering an opening in the body distal end.

20. The cartridge of claim 19, wherein the seal is frangible and permanently coupled to the body distal end, so that a pharmaceutical formulation passage can be formed upon mating a piercing device to the body distal end without removing the seal.

21. The cartridge of claim 18, wherein an outer surface of the body distal end is tapered.

22. The cartridge of claim 18, wherein the body is a glass tube.

23. The cartridge of claim 18, further comprising a pharmaceutical formulation having a viscosity of from about 100 to about 500,000 poise.

24. The cartridge of claim 23, wherein the pharmaceutical formulation has a viscosity of from about 1,000 to about 5,000 poise.

25. A method of delivering a pharmaceutical formulation, comprising the steps of:
a) providing a cartridge in accordance with claim 1;
b) engaging the plunger with a separately formed rod;
c) transferring the plunger from a first position to a second position by applying a normal force to the rod, such that the pharmaceutical formulation is expelled from the cartridge.

26. The method of claim 25, wherein the plunger includes a recess formed therein for receiving the rod.

27. The method of claim 25, wherein the bore has a transverse dimensions that is constant along its entire length.

28. A method of delivering a pharmaceutical formulation, comprising the steps of:
a) providing a cartridge comprising:
i) a body including a bore extending through the body, and an end surface having an opening therein that is in fluid communication with the bore;
ii) a pharmaceutical formulation disposed within a portion of the bore, and
iii) a plunger movably disposed within the bore for expelling the pharmaceutical formulation;
b) transferring the plunger from a first position to a second position, such that a contact surface of the plunger in the second position is flush with, or extends slightly beyond, the body end surface.

29. The method of claim 28, wherein the bore has a constant transverse dimension along its entire length.

30. The method of claim 28, wherein the cartridge further comprises a seal covering the opening in the body end surface.

31. The method of claim 28, wherein the pharmaceutical formation has a viscosity of from about 100 to about 500,000 poise.

32. The method of claim 31, wherein the pharmaceutical formulation has a viscosity of from about 1,000 to about 5,000 poise.

33. The method of claim 28, wherein the body has a tapered outer surface at its distal end.

34. The method of claim 28, wherein the plunger is devoid of any member extending beyond the body.

35. The method of claim 28, wherein an engagement end of the plunger includes a recess for accepting a rod.

36. The method of claim 28, wherein the step of transferring the plunger from the first position to the second position includes engaging the plunger with a separately formed rod and applying a normal force to the rod.
